(19) United States
(12) Patent Application Publication
(10) Pub. No.: US 2021/0307750 A1
(43) Pub. Date: Oct. 7, 2021

(54) SURGICAL STAPLER AND RELATED METHODS

(71) Applicant: Bolder Surgical, LLC, Louisville, CO (US)

(72) Inventors: Allison Lyle, Boulder, CO (US); Richard Granger, Niwot, CO (US); Nathan Steketee, Superior, CO (US); Joseph Bucciaglia, Boulder, CO (US); Jenifer Kennedy, Boulder, CO (US)

(21) Appl. No.: 17/413,650

(22) PCT Filed: Dec. 17, 2019
(86) PCT No.: PCT/US2019/066804

§ 371 Date: Jun. 14, 2021

Related U.S. Application Data
(60) Provisional application No. 62/781,074, filed on Dec. 18, 2018.

(51) Int. Cl. A61B 17/072; A61B 17/064

(52) U.S. Cl. CPC A61B 17/07207; A61B 17/0644; A61B 2017/07278; A61B 2017/07271; A61B 2017/07257; A61B 2017/07285

(57) ABSTRACT

A surgical staple, stapler, and related methods are disclosed. A surgical staple has a pair of staple legs and a backspan positioned between the pair of staple legs, the backspan and the pair of staple legs formed by bending a wire. At least one recess is positioned in the wire to influence a folding direction of at least one staple leg during tissue stapling. The surgical staple is configured to pierce tissue only once with each staple leg during the tissue stapling.

### CROSS-REFERENCE TO RELATED APPLICATIONS

This application is a national phase entry of, and claims priority to, PCT Application No. PCT/US19/66804, filed Dec. 17, 2019 and entitled “SURGICAL STAPLER AND RELATED METHODS,” which claims priority to U.S. Provisional Application No. 62/781,074, filed Dec. 18, 2018 and entitled “SURGICAL STAPLER AND RELATED METHODS,” the entire disclosures of which are hereby incorporated by reference for all proper purposes.

### FIELD OF THE INVENTION

This invention is related to surgical staplers. Specifically, but not intended to limit the invention, embodiments of the invention are related to staplers, actuators, and staples.

### BACKGROUND OF THE INVENTION

A number of surgical staplers are known in the industry. In some designs, a cartridge is provided to support a number of staples therein. An anvil rotatable relative to the cartridge is also provided to assist with staple formation during staple deployment. There remains a need, however, for a stapler and methods that provide better control during deployment and/or other new and useful improvements.

### SUMMARY OF THE INVENTION

An exemplary surgical staple has a pair of staple legs and a backspan positioned between the pair of staple legs, the backspan and the pair of staple legs formed by bending a wire. At least one recess is positioned in the wire to influence a folding direction of at least one staple leg during tissue stapling. The exemplary surgical staple is configured to pierce tissue only once with each staple leg during the tissue stapling.

An exemplary method of making a surgical staple includes providing a wire, and bending the wire to form a pair of staple legs and a backspan positioned between the pair of staple legs. The exemplary method includes forming at least one recess positioned in the wire to influence a folding direction of at least one staple leg during tissue stapling, wherein the surgical staple is configured to pierce tissue only once with each staple leg during the tissue stapling. The forming the at least one recess includes at least one of displacing material in the wire or removing material from the wire. The surgical staple is configured to form a consistent tissue gap between the pair of staple legs and the backspan after the tissue stapling.

An exemplary method includes providing a surgical stapler. The surgical stapler has an anvil opposing a cartridge, the cartridge housing at least one staple, the at least one staple having a pair of staple legs, a backspan positioned between the pair of staple legs, the backspan and the pair of staple legs formed by bending a wire, and at least one recess positioned in the wire to influence a folding direction of at least one staple leg during tissue stapling. The surgical staple is configured to pierce tissue only once with each staple leg during the tissue stapling. The exemplary method further includes approximating the anvil and the cartridge with tissue positioned therebetween. The exemplary method further includes ejecting the at least one staple from the cartridge toward the anvil to cause the pair of staple legs pierce the tissue and to contact the anvil and bend the pair of staple legs inward to form the consistent tissue gap without piercing the tissue a second time.

### BRIEF DESCRIPTION ON THE DRAWINGS

FIG. 1 is a perspective view of a surgical stapler in an open configuration;

FIG. 2 is a side view of the stapler in FIG. 1;

FIG. 3 is a perspective view of an end effector for the stapler in FIG. 1;

FIG. 4 is an exploded perspective view of the stapler in FIG. 1;

FIG. 5 is a perspective view of the stapler in FIG. 1 in a trocar configuration;

FIG. 6 is side view of the stapler in FIG. 4;

FIG. 7 is a side view illustrating portions of an I-beam suitable for use in the stapler in FIG. 1;

FIG. 8 is a side section view of sections of the stapler in FIG. 1 in a home configuration;

FIG. 9 is a side section view of the components in FIG. 8 in a pre-stapling configuration;

FIG. 10 is a side section view of the components in FIG. 8 in a post-stapling configuration;

FIG. 11 is a side section view of the components in FIG. 8 in a post-stapling I-beam retracted configuration;

FIG. 12 is a perspective view of an I-beam suitable for use in the stapler in FIG. 1;

FIG. 13 is a perspective view of the I-beam in FIG. 12 in a detached state;

FIG. 14 is a perspective view of an I-beam suitable for use in the stapler in FIG. 1;

FIG. 15 is a top view of an anvil suitable for use in the stapler in FIG. 1;

FIG. 16 is a top view of a proximal portion of the anvil in FIG. 15;

FIG. 17 is a side section view illustrating portions of a stapler in a home configuration;

FIG. 18 is a side section view of components illustrated in FIG. 17;

FIG. 19 is a side view of components illustrated in FIG. 17;

FIG. 20 is a side section view of components illustrated in FIG. 17;

FIG. 21 is a side section view of components illustrated in FIG. 17;

FIG. 22 is a side section view of components illustrated in FIG. 17;

FIG. 23 is a side view of components illustrated in FIG. 17;

FIG. 24 is a side section view of components illustrated in FIG. 17;

FIG. 25 is a side section view of components illustrated in FIG. 17;

FIG. 26 is a perspective view of a surgical staple;

FIG. 27 is a side view of the staple in FIG. 26;

FIG. 28 is a side view of a staple after deformation;

FIG. 29 is a perspective view of a surgical staple;

FIG. 30 is a side view of the surgical staple in FIG. 28;

FIG. 31 is an end view of the surgical staple in FIG. 28;

FIG. 32 is a side view of an exemplary staple;

FIG. 33 is a side view of an exemplary staple; and

FIG. 34 is a side view of the staple in FIG. 32 after deformation.

### DETAILED DESCRIPTION

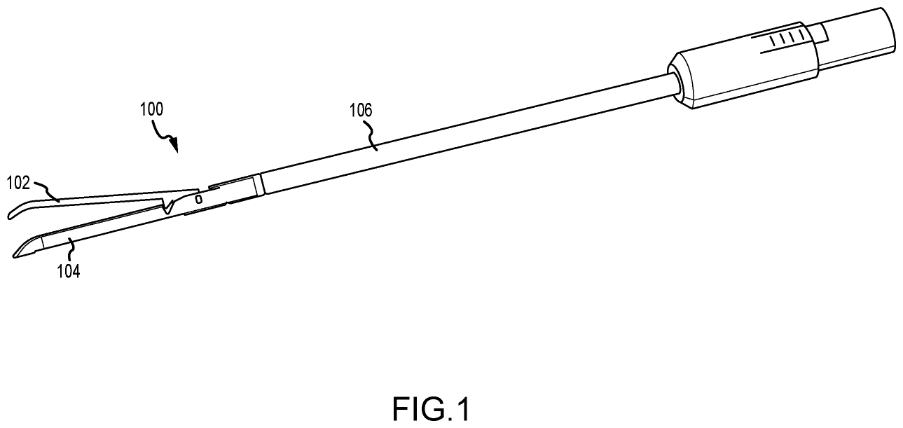

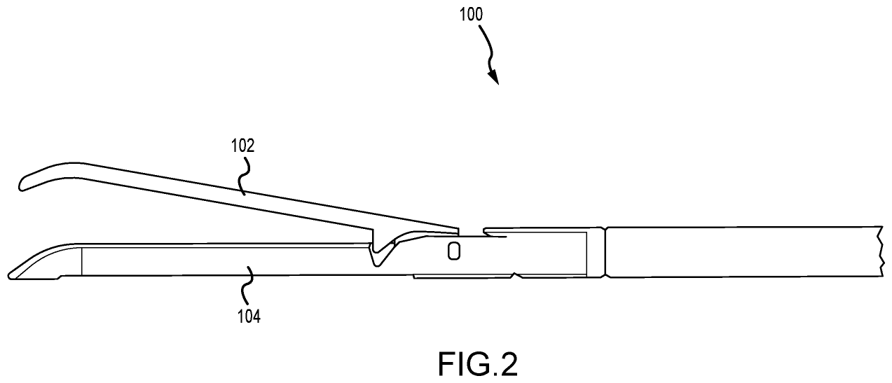

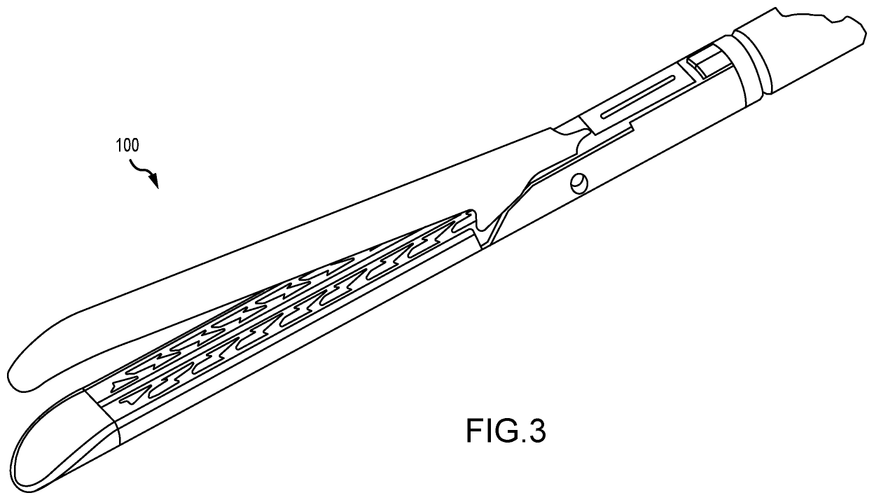

With reference now to FIGS. 1-3, a surgical stapler 100 is now described. The stapler 100 may have an upper jaw or anvil 102, and a lower jaw 104, together forming an end effector, coupled to the distal end of an elongated shaft 106. One or both jaws 102, 104 may be movable relative to the other jaw(s) 102, 104 between a trocar configuration, an open configuration, and a tissue clamp configuration, which will be described in subsequent portions of this document.

One of the jaws 104 may house a plurality of staples (not illustrated) therein, for translation towards the other jaw 102 and deformation to staple tissue clamped between the jaws 102, 104. Those skilled in the art will recognize that the jaw 104 may include a housing and cartridge for holding the staples, or, as illustrated, the jaw 104 may be a single component for housing the staples and/or pushers and operatively couple to the elongated member 106.

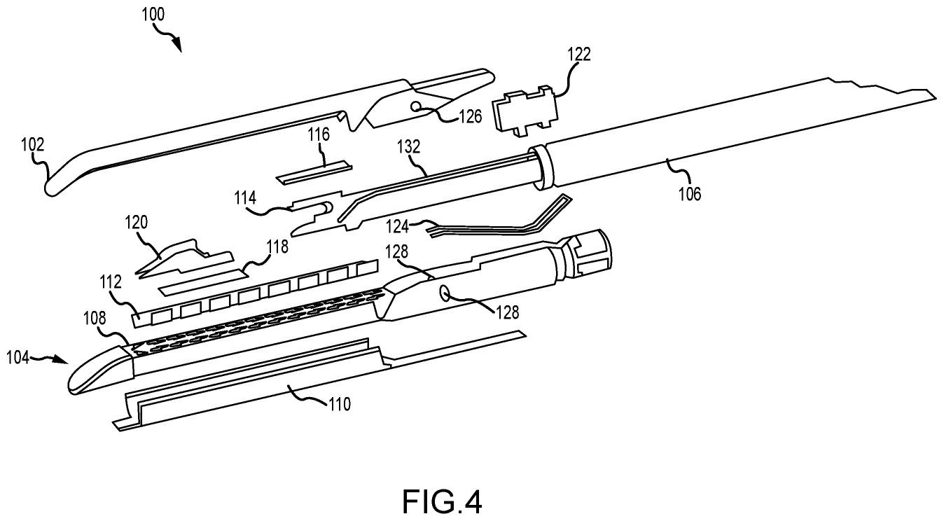

As illustrated in FIG. 4, the stapler 100 may include an upper jaw or anvil 102, a lower jaw 104 having a cartridge 108 housing staple pushers 112 (and staples, not illustrated) and a slide plate 110. Those skilled in the art will readily recognize that the terms “cartridge housing” or “housing” may be used interchangably with the term “slide plate”. The stapler 100 may also include an elongated member 114 for translation to cut tissue clamped between the jaws 102, 104. The elongated member 114 may include or otherwise engage upper clamping member 116 and lower clamping member 118 during a clamping action on the jaws 102, 104. The member 114 may also engage a sled 120 during a stapling action, such as to cause the staple pusher(s) 112 to translate. A spring insert 122 and/or spring 124 may be provided to assist in opening and/or closing the jaws 102, 104 in a manner as described herein.

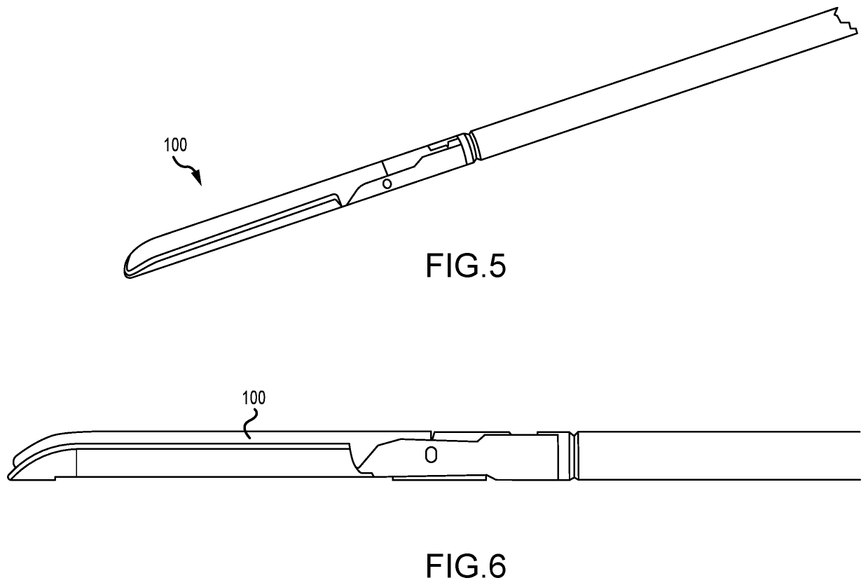

FIG. 5 and FIG. 6 illustrate the stapler 100 in a trocar configuration, as described in further detail herein.

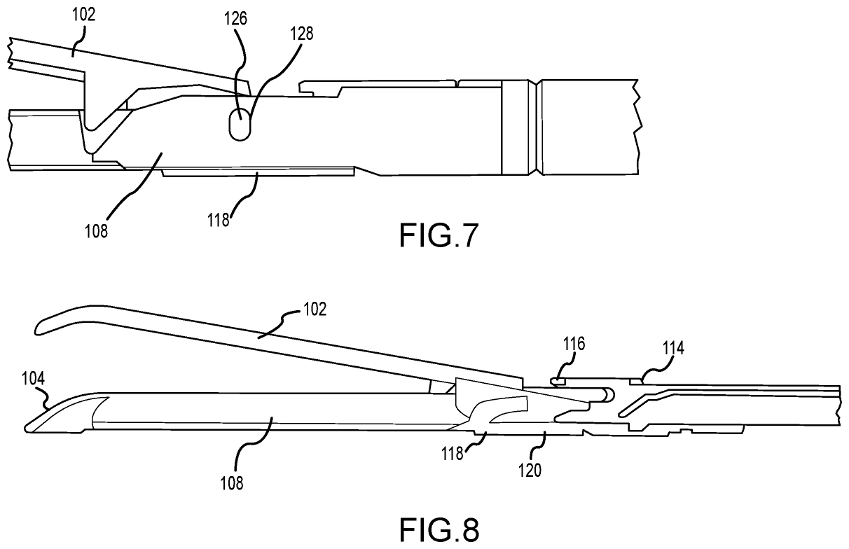

FIG. 7 and FIG. 8 illustrate details of the stapler 100 in a home or open configuration, and operation of the stapler 100 is now described in further detail. Starting in the home configuration, a distal portion of the anvil 102 is moved or rotated away from the cartridge or jaw 104, while a proximal portion of the anvil 102 is compressed against the jaw 104. That is, a pin 126 in the anvil 102 may slidably and rotatably engage an elongated slot in the jaw 104 or cartridge 108, and, in the home position, the pin 126 may engage a lower portion of the elongated slot 128 while the distal portion of the anvil 102 is spaced apart from the jaw 104. In some embodiments, and as most clearly seen in FIG. 4 and FIG. 15, a pair of pins 126 in the anvil 102 may engage a pair of slots 128 in the jaw 104 or cartridge 108 to limit translational motion of the proximal portion of the anvil 102. Further details of this function can be found in co-owned U.S. Pat. No. 10,143,474, which issued on Dec. 4, 2018 to Bucciaglia et al.

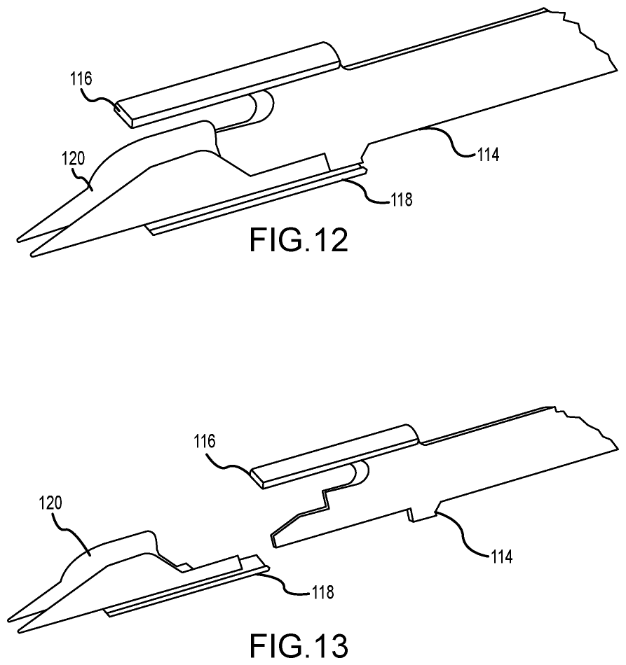

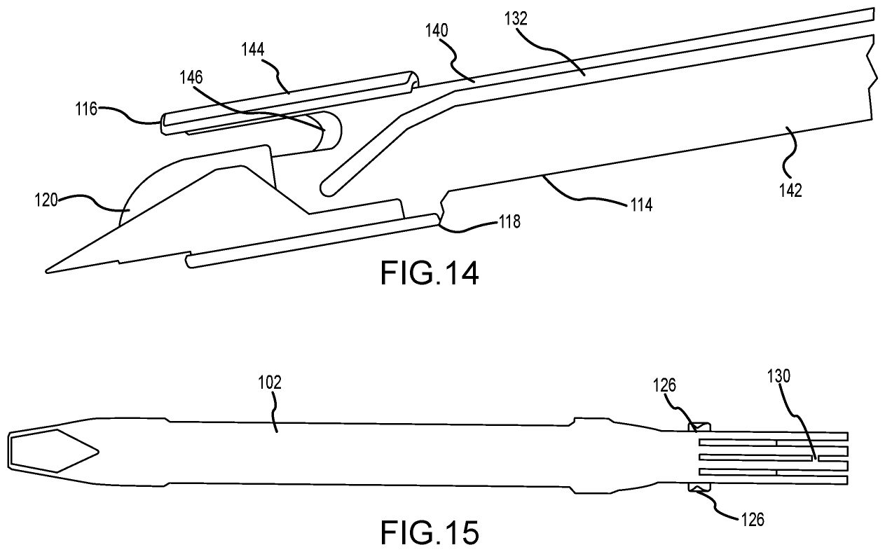

Continuing with FIG. 7 and FIG. 8, and with simultaneous reference to FIG. 12, FIG. 13, and FIG. 14, the elongated member 114 is configured to push a sled 120 to engage staple pushers 112 to fire staples (not illustrated). The sled 120 may be permanently affixed to the lower clamping member 118, which may provide tissue compression and gap control during staple firing. When the elongated member 114 completes a stroke, it may be retracted, leaving the sled 120 and lower clamping member 118 in a distal portion of the stapler 100. In some embodiments, one or more detents (not illustrated) may be positioned in a distal portion of the cartridge 108, slide wall 110, or jaw 104 to capture the sled 120 in the distal portion, although other means of capturing the sled 120 are contemplated.

By disconnecting the lower clamping member 118 prior to retracting the elongated member 114, the elongated member 114 does not clamp the jaws 102, 104 of the device together during a retracting motion. One advantage of disconnecting the lower clamping member 118 prior to retracting is that the force required to retract the elongated member 114 may be reduced, which can be a major driver of handle design in some surgical staplers. For example, some surgical staplers currently on the market include a separate lever that the user must retract (using a second hand) to pull the member back. Others require an additional trigger actuation to retract the member. Still others rely on a motor to drive and/or retract the member.

Another advantage of disengaging the lower member 118 from the elongated member 114 is that, in the event the stapler 100 malfunctions or is fired upon an obstruction and/or the member 114 seizes mid-stroke, the user has the ability to disengage the member 114 from the lower clamp or jaw 104 and release the tissue. With existing staplers, if the drive member jams in the middle of the stroke and cannot be retracted, the tissue must be cut out of the jaws.

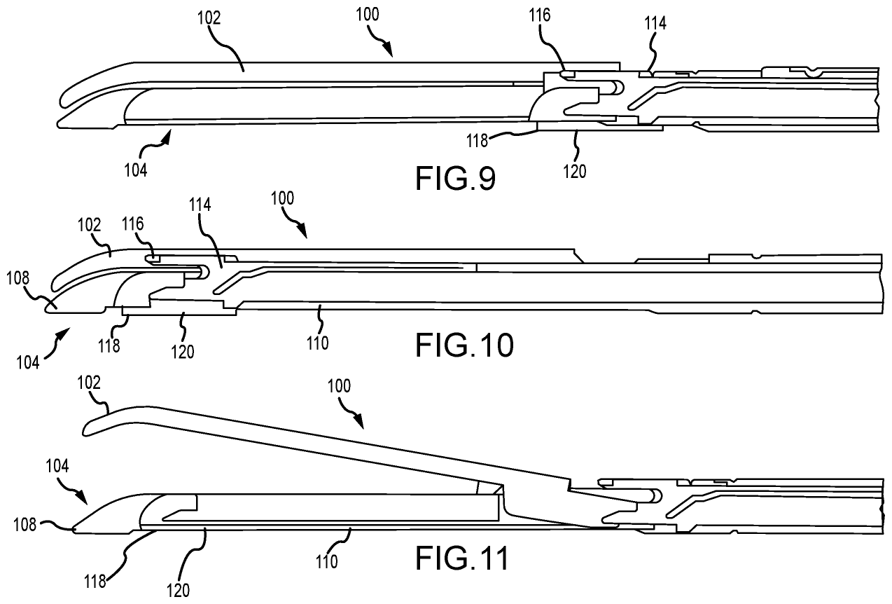

FIG. 9 and FIG. 10 illustrate an example of the member 114 as it is moved distally along with the sled 120 and the upper clamping member 116 and the lower clamping member 118. FIG. 11 illustrates the sled 120 and the lower clamping member 118 detached and positioned in the distal portion of the stapler 100 after the elongated member 114 is retracted with the upper clamping member 116. Those skilled in the art will recognize that, although this document describes upper and lower clamping members 116, 118, with particular features and the lower clamping member 118 detaching, the situation could be reversed, with the upper clamping member 116 being parked in the distal portion of the anvil 102. Those skilled in the art will also recognize that the elongated member 114 illustrated in FIGS. 9-11 having a slot (as will be described in further detail herein) for assisting in manipulating the jaws 102, 104, this feature is not necessary to the functioning of the upper and lower clamping member 116, 118 and the detachable sled 120 and/or detachable lower clamping member 118. See, e.g., FIG. 12 and FIG. 13 illustrating the member 114 without a slot.

In some embodiments, the stapler 100 may include a bailout mechanism (not illustrated) to provide the user with the ability to extract the member 114 if it seizes mid-stroke.

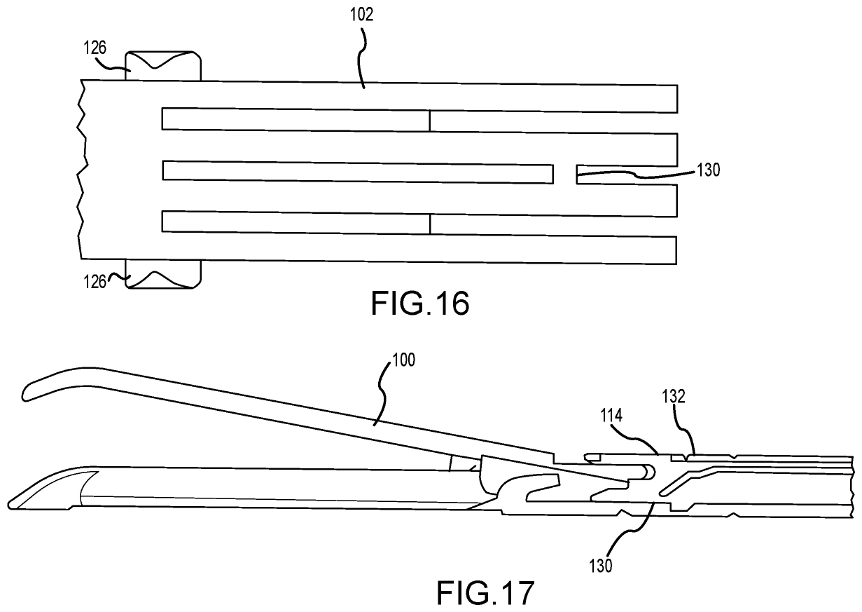

Turning now to FIGS. 14-25, in some embodiments, the elongated member 114 may include a slot 132 to engage a transverse member 130 on the anvil 102 to control the anvil 102. For example, the transverse member 130 may be positioned in a proximal portion of the anvil 102, and the elongated member 114 may actuate the anvil 102 to rotate and translate within a vertical pivot slot 128. In some embodiments, the member 114 may open the anvil 102 as the member 114 moves proximally. Distal motion of the member 114 may close the anvil 102. Closing the anvil 102 may cause the anvil to pass through three distinct configurations: Position 1: open, Position 2: zero gap/trocar position, Position 3: tissue gap/clamp position. The configurations are described in further detail below. Actuating the anvil 102 in this manner eliminates the need for an additional actuator to effectuate anvil movement.

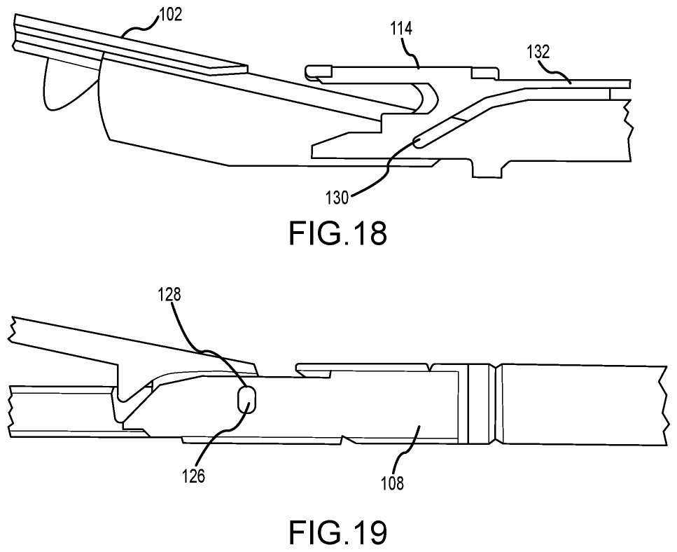

As illustrated in FIG. 17 and FIG. 18, when the member 114 is pulled proximally, the anvil pin or transverse member 130 is forced or cammed downward in the slot 132 to open the anvil 102 or place the stapler 100 in the first configuration. The anvil 102 is at the bottom of its pivot slot(s) 128, such that the anvil pin(s) 126 abut a lower portion of the slots 128, as illustrated in FIG. 19.

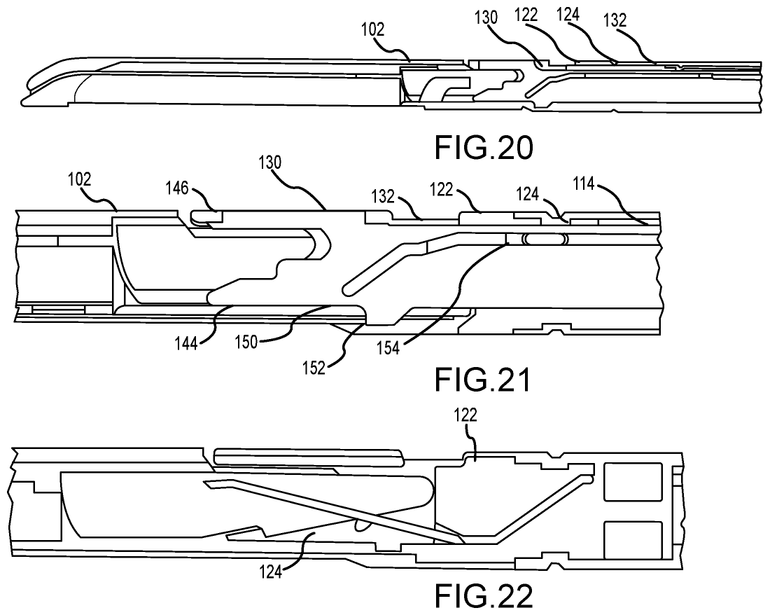

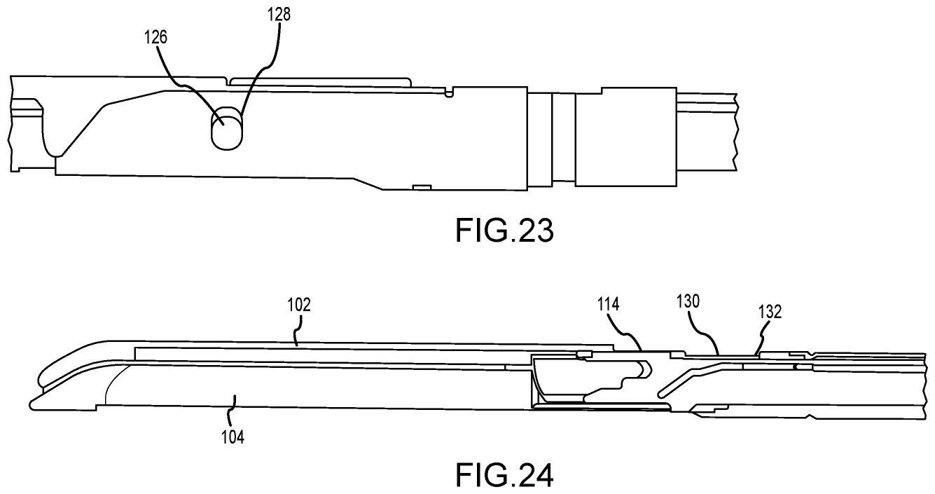

Turning now to FIG. 20, when the member 114 is pushed distally towards or into position 2, the pin or transverse member 130 in the anvil 102 slides higher in the slot 132 to a position where the anvil 102 is positioned to pass through a trocar (zero tissue gap). As illustrated in FIG. 21 and FIG. 22, a spring 124 and/or elastomer 122 pushes the anvil 102 downward in a pivot slot 128 in the lower jaw 104 or cartridge 108 to encourage the anvil 102 to this position, wherein the stapler 100 is in a trocar configuration or second configuration. See also FIG. 23. Those skilled in the art will also recognize that the slot 132 may include an angle between position 1 and position 2. The angle may be selected to control the linear stroke required to move between position 1 and position 2.

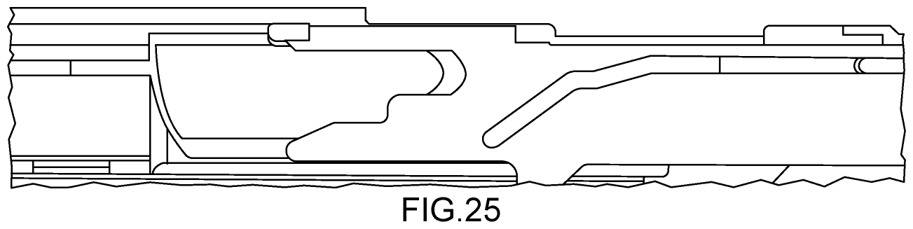

Turning now to FIG. 24 and FIG. 25, when the member 114 is pushed further distally to position 3, the pin or transverse member 130 in the anvil 102 is pushed further upward in the slot 132, which applies clamp force at the distal end of the anvil 102. FIG. 24 illustrates the stapler 100 in a tissue clamp configuration or third configuration.

Returning now to FIG. 14, in some embodiments, a cutting mechanism 140 for a surgical stapler such as the stapler 100 illustrated in FIG. 1, may include, an elongated member 114 having a length and a height, a proximal portion 142, a distal portion 144, and an elongated slot 132 extending a portion of the length of the elongated member 114. The distal portion 144 may have a cutting surface 146, such as for severing tissue clamped between jaws 102, 104 of the stapler 100. The elongated slot 132 may have a distal slot portion 150, an intermediate slot portion 152, and a proximal slot portion 154. The distal slot portion 150 may be one of higher or lower than the intermediate slot portion 152. The proximal slot portion 154 may be positioned the other one of higher or lower than the intermediate slot portion 152. In some embodiments, a vertical distance is provided between the distal slot portion 150 and the intermediate slot portion 152. The vertical distance may be a function of a desired tissue gap. The desired tissue gap may be more than, less than, or equal to the gap that the I-beam 114 imposes on the housing and anvil during deployment. An upper clamping member 116 may be adjacent the distal portion 144 of the elongated member 114. A lower clamping member 118 may be adjacent the distal portion 144 of the elongated member 114. As illustrated in FIG. 13, one of the upper clamping member 116 or the lower clamping member 118 may be detachable from the elongated member 114, and the other one of the upper clamping member 116 or the lower clamping member 118 may be fixed to the elongated member 114.

In some embodiments, a surgical stapler has a cutting mechanism as described above, and a sled 120 for ejecting staples, wherein the sled 120 is translated distally by the elongated member 114, and wherein the sled 120 and the lower clamping member 118 are deposited in a distal portion of the stapler during staple ejection.

A method of operating a surgical stapler may include (1) positioning a cutting mechanism in a proximal position whereby the stapler is placed in an open configuration; (2) positioning the cutting mechanism in an intermediate position whereby the stapler is placed in a trocar configuration; and (3) positioning the cutting mechanism in a distal position whereby the stapler is placed in a tissue clamp configuration.

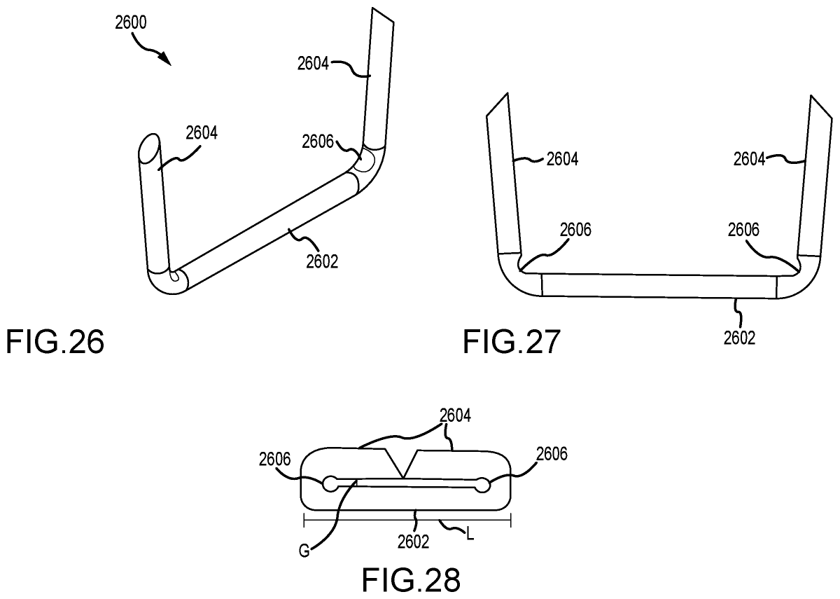

Turning now to FIGS. 26-28, a surgical staple is described. In some embodiments, a staple 2600 may have a backspan 2602 positioned between a pair of staple legs 2604. The legs 2604 may form an angle to the backspan. The backspan 2602 and the legs 2604 may be formed by bending a wire or a metallic component as is known in the art.

In some embodiments, the backspan 2602 has a length L. The length L may be substantially in line with an intended staple line. The length L may be greater than a width of the backspan 2602. In some embodiments, the length L is greater than about 3 millimeters. In some embodiments, the length is about 4 millimeters or more. In some embodiments, the length L is about 5 millimeters. In some embodiments, the length L of the backspan 2602 is greater than a length of the leg(s) 2604.

At the bend between the backspan 2602 and one or both legs 2604 may be a recessed portion 2606. The recessed portion 2606 may be a portion that is notched out of the material forming the backspan 2602 and legs 2604, and may be referenced herein as a notch. The notch may be formed by displacing material (such as by a coining operation) or by removing material.

In some embodiments, the recessed portion 2606 influences the folding direction of the associated staple leg 2604. The recessed portion 2606 may reduce the incidence of a leg buckling and/or may reduce the force required to bend the leg 2604 relative to the backspan 2602.

In some embodiments, the recessed portions 2606 are placed to cause the leg(s) 2604 to form inward (e.g. in plane or parallel with with the backspan 2602) after stapling.

In some embodiments, the recessed portions 2606 may be placed circumferentially around the leg(s) 2604 to form the legs around (straddling) the backspan 2602.

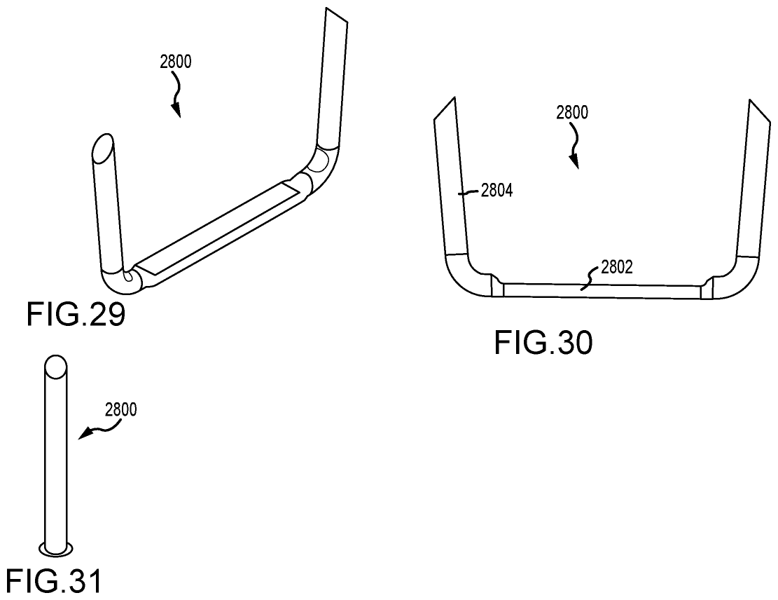

Turning now to FIGS. 29-31, in some embodiments, a staple 2800 may include a flattened backspan 2802 and rounded leg(s) 2804. The combination of round and flat wire may better accommodate tissue variability. For example, if the stapled tissue is thick, it can displace to the region where the backspan 2802 is flat and the staple gap is larger. If the tissue is thin relative to the formed staple gap, the staple legs 2804 will appose the tissue walls and the rounded corners can pinch and secure it. The flat backspan also has more contact area with the tissue so it may not slide around on the tissue.

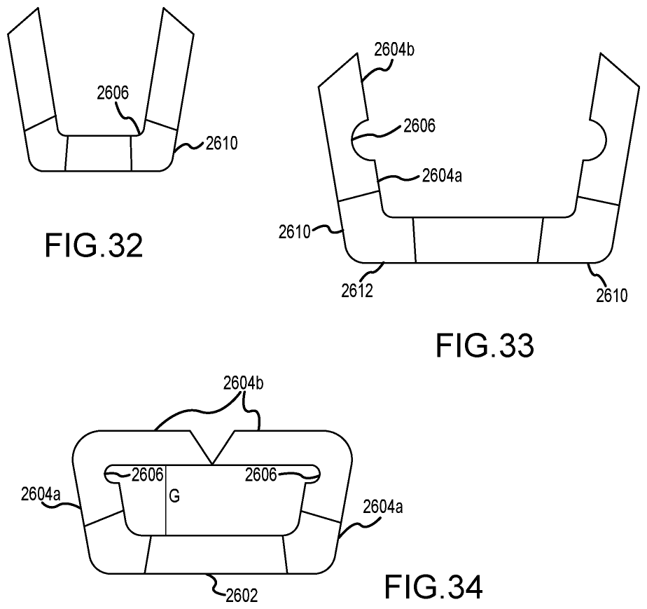

As illustrated in FIG. 32, in some embodiments, the staple 2600 may include a texturized surface 2610. The texturized surface 2610 may be positioned and configured to promote clotting around the portion of the staple that remains in contact with stapled tissue. For example, the texturized surface 2610 may be a surface proximal to a location where the backspan 2602 transitions to a leg 2604. In some embodiments, one or both legs 2604 may include a proximal leg portion 2604a proximal to the backspan 2602 and a distal leg portion 2604b distal of the backspan 2602, wherein a portion or all of the proximal leg portion 2604a has a texturized surface 2610 and the distal leg portion 2604b is not texturized. In some embodiments, the proximal leg portion 2604a has a greater envelope cross section than does the distal leg portion 2604b, so as to ensure that the proximal leg portion 2604a remains in contact with tissue after the stapling procedure. In some embodiments, the distal leg portions 2604b and a majority of the surface of the backspan 2602 are not texturized.

In some embodiments, a portion or all of the surface area intended for contact with stapled tissue may be texturized. In some embodiments, the texturized surface 2610 is provided by way of mechanical treatment (such as, for example, knurling, coining, blasting), chemical treatment (such as, for example, etching), and/or other energy treatment (such as, for example, EDM, laser).

In some embodiments, and as illustrated in FIG. 33, a portion or all of the staple 2600 may be coated with a clotting agent 2612, which may be referenced herein as a coagulant. In some embodiments, the texturized surface(s) 2610 is partially or fully coated with a clotting agent 2612. The clotting agent 2612 may include an active absorbable collagen hemostat, such as, for example only, that marketed by C.R. Bard under the brand name Avitene™, to accelerate clot formation. The clotting agent 2612 may be provided to enhance platelet aggregation and/or the release of proteins to form fibrin. The clotting agent 2612 may include hemostatic granules, such as the granules marketed by Celox Medical under the brand name Celox™, and/or other clotting agents, such as, for example, silver nitrate.

As illustrated in FIG. 33 and FIG. 34, the recessed portion(s) 2606 may be placed a distance from the point of joinder between the backspan 2602 and leg(s) 2604. That is, the position of the recessed portion 2606 on the leg 2604 and/or notch size may be controlled to achieve a desired gap G between the formed staple leg and backspan. In some embodiments, the recessed portion 2606 is positioned between a proximal leg portion 2604a and a distal leg portion 2604b.

In some embodiments, the staple 2600 may be configured to pierce tissue only once with each staple leg 2604. That is, the staple 2600 may be configured so as to prevent the staple 2600 from piercing stapled tissue a second time with the same leg 2604 such as by rolling the staple leg 2604 back. It is believed by the Applicant that limiting tissue piercing is particularly beneficial when stapling on vasculature.

Returning now to FIG. 28, the staple 2600 may be configured to form a flattened staple shape with a gap of less than about 0.012 inches (0.3048 millimeters) or less than 0.31 millimeters between any portion of the staple leg 2604 and the backspan 2602 after stapling. In some embodiments, the gap is greater than 0.002 inches (0.0508 millimeters) and less than about 0.006 inches (0.1524 millimeters). In some embodiments, the gap G is between about 0.004 inches (0.1016 millimeters) and about 0.006 inches (0.1524 millimeters). In some embodiments, the gap G is about 0.005 inches (0.127 millimeters). In some embodiments, the gap is between 0.09 millimeters and 0.16 millimeters. In some embodiments, the gap is less than about 0.004 inches (0.1016 millimeters) or less than 0.12 millimeters. The resulting ‘flattening’ of the staple legs, which provides a consistent tissue gap and applies even distribution of clamp pressure across the tissue, as opposed to an arched leg formation with a variable tissue gap.

In some embodiments, and as illustrated in FIG. 34, the staple 2600 may be configured with a gap G that is greater than 0.012 inches (0.3048 millimeters), such as when the joinder of thick tissue is desired. In some embodiments, the leg(s) 2604 may have a proximal leg portion 2604a and a distal leg portion 2604b configured to provide a tissue gap G. The tissue gap G may be greater than 0.012 inches (0.3048 millimeters) in some embodiments. The tissue gap G may be less than 0.012 inches (0.3048 millimeters) in some embodiments. The tissue gap G may be greater than 0.006 inches (0.1524 millimeters) in some embodiments. The tissue gap G may be less than 0.006 inches (0.1524 millimeters) in come embodiments. The tissue gap G may be between about 1 millimeter and about 2.5 millimeters for some thick tissue applications. Those skilled in the art will recognize that placement of the recess(es) 2606 will control the size of the gap G. Those skilled in the art will recognize that the size of the gap G and the staple leg length may be related.

The following clauses set out further illustrative aspects useful for understanding the appended claims:

Clause (1): A cutting mechanism for a surgical stapler, the cutting mechanism having: an elongated member having a length and a height, a proximal portion, a distal portion, and an elongated slot extending a portion of the length of the elongated member, wherein the distal portion comprises a cutting surface, and wherein the elongated slot comprises a distal slot portion, an intermediate slot portion, and a proximal slot portion, the distal slot portion positioned one of higher or lower than the intermediate slot portion, and the proximal slot portion positioned the other one of higher or lower than the intermediate slot portion; and a first clamping member adjacent the distal portion of the elongated member.

Clause (2) The cutting mechanism of clause 1, wherein: the distal portion is configured to detachably engage a second clamping member adjacent the distal portion of the elongated member.

Clause (3) The cutting mechanism of clause 2, wherein: the first clamping member is an upper clamping member fixed to the elongated member; and the second clamping member is a lower clamping member; and wherein the upper clamping member and the lower clamping member are configured to limit a space between the upper and lower clamping members.

Clause (4) The cutting mechanism of clause 1, wherein: the second clamping member further comprises a sled configured to engage one or more staple pushers.

Clause (5) A surgical stapler comprising: a cartridge housing at least one staple; an anvil movable relative to the cartridge a cutting mechanism as claimed in claim 1, the cutting mechanism movable between a proximal position and a distal position relative to the cartridge; and a sled for ejecting the at least one staple; wherein the elongated member is configured to engage the sled during a first movement from the proximal position to the distal position, whereby the at least one staple is ejected during the first movement.

Clause (6) The surgical stapler of clause 5, wherein: the elongated member is configured to disengage from the sled during a second movement from the distal position to the proximal position, whereby the sled is deposited at a distal location in the cartridge.

Clause (7) The surgical stapler of clause 5, wherein: the first clamping member of the cutting mechanism is an upper clamping member fixed to the elongated member; and the second clamping member is a lower clamping member; and wherein the upper clamping member and the lower clamping member are configured to limit a space between the upper and lower clamping members.

Clause (8) The surgical stapler of clause 5, wherein: the sled is fixed to the second clamping member.

Clause (9) The surgical stapler of clause 5, further comprising: a resilient member configured to bias the anvil one of toward the cartridge or away from the cartridge.

Clause (10) The surgical stapler of clause 5, further comprising: a resilient member configured to bias the anvil toward the cartridge.

Clause (11) The surgical stapler of clause 9 or 10, wherein: the resilient member and the first clamping member cooperate to position the anvil in a tissue clamp configuration when the elongated member is in an intermediate position between the proximal position and the distal position.

Clause (12) A method of operating a surgical stapler having an anvil movable relative to a staple cartridge, the method comprising: positioning a cutting mechanism in a proximal position wherein the cutting mechanism engages the anvil at a first location on the cutting mechanism and whereby the stapler is placed in an open configuration wherein the anvil and the cartridge are in a spaced apart configuration; positioning the cutting mechanism in an intermediate position wherein the cutting mechanism engages the anvil at a second location on the cutting mechanism and whereby the stapler is placed in a trocar configuration wherein the anvil and the cartridge are fully clamped together; and positioning the cutting mechanism in a distal position wherein the cutting mechanism engages the anvil at a third location on the cutting mechanism and whereby the stapler is placed in a tissue clamp configuration wherein the anvil and the cartridge are substantially parallel to each other and have a tissue gap therebetween.

Clause (13) The method of clause 12, wherein: the first location is distal of and lower than the second location and the third location; and the second location is distal of and lower than the third location.

Each of the various elements disclosed herein may be achieved in a variety of manners. This disclosure should be understood to encompass each such variation, be it a variation of an embodiment of any apparatus embodiment, a method or process embodiment, or even merely a variation of any element of these. Particularly, it should be understood that the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which this invention is entitled.

As but one example, it should be understood that all action may be expressed as a means for taking that action or as an element which causes that action. Similarly, each physical element disclosed should be understood to encompass a disclosure of the action which that physical element facilitates. Regarding this last aspect, the disclosure of a “fastener” should be understood to encompass disclosure of the act of “fastening”—whether explicitly discussed or not—and, conversely, were there only disclosure of the act of “fastening”, such a disclosure should be understood to encompass disclosure of a “fastening mechanism”. Such changes and alternative terms are to be understood to be explicitly included in the description.

Moreover, the claims shall be construed such that a claim that recites “at least one of A, B, or C” shall read on a device that requires “A” only. The claim shall also read on a device that requires “B” only. The claim shall also read on a device that requires “C” only.

Similarly, the claim shall also read on a device that requires “A+B”. The claim shall also read on a device that requires “A+B+C”, and so forth.

The claims shall also be construed such that any relational language (e.g. perpendicular, straight, parallel, flat, etc.) is understood to include the recitation “within a reasonable manufacturing tolerance at the time the device is manufactured or at the time of the invention, whichever manufacturing tolerance is greater”.

Those skilled in the art can readily recognize that numerous variations and substitutions may be made in the invention, its use and its configuration to achieve substantially the same results as achieved by the embodiments described herein.

Accordingly, there is no intention to limit the invention to the disclosed exemplary forms. Many variations, modifications and alternative constructions fall within the scope and spirit of the invention as expressed in the claims.

What is claimed is:

1. A surgical staple, comprising:
a pair of staple legs;
a backspan positioned between the pair of staple legs, the backspan and the pair of staple legs formed by bending a wire; and
at least one recess positioned in the wire, the at least one recess configured to influence a folding direction of at least one staple leg during tissue stapling; and wherein
the surgical staple is configured to pierce tissue only once with each staple leg during the tissue stapling.

2. The surgical staple of claim 1,
further configured to form a consistent tissue gap between the pair of staple legs and the backspan after the tissue stapling.

3. The surgical staple of claim 2, wherein:
the at least one recess is shaped and positioned to influence a size of the consistent tissue gap.

4. The surgical staple of claim 2, wherein:
the consistent tissue gap is less than 0.3048 millimeters.

5. The surgical staple of claim 2, wherein:
the consistent tissue gap is greater than 0.3048 millimeters.

6. The surgical staple of claim 1, wherein:
the at least one recess is positioned between the backspan and a distal portion of a respective staple leg.

7. The surgical staple of claim 1, wherein:
the backspan further comprises a flat surface positioned to engage tissue after the tissue stapling.

8. The surgical staple of claim 1, further comprising:
a texturized surface for promoting clotting of stapled tissue after the tissue stapling.

9. The surgical staple of claim 1, further comprising:
a clotting agent coating at least a portion of the surgical staple.

10. A method of making a surgical staple, the method comprising:
providing a wire;
bending the wire to form a pair of staple legs and a backspan positioned between the pair of staple legs; and
forming at least one recess positioned in the wire, the recess configured to influence a folding direction of at least one staple leg during tissue stapling, wherein the surgical staple is configured to pierce tissue only once with each staple leg during the tissue stapling; wherein
the forming the at least one recess comprises at least one of displacing material in the wire or removing material from the wire; and wherein
the surgical staple is configured to form a consistent tissue gap between the pair of staple legs and the backspan after the tissue stapling.

11. The method of claim 10, wherein
the shaping and positioning the at least one recess comprises shaping and positioning the at least one recess to influence a size of the consistent tissue gap.

12. The method of claim 10, wherein:
the consistent tissue gap is less than 0.3048 millimeters.

13. The method of claim 10, wherein:
the consistent tissue gap is greater than 0.3048 millimeters.

14. The method of claim 10, further comprising:
positioning the at least one recess between the backspan and a distal portion of a respective staple leg.

15. The method of claim 10, further comprising:
providing a flat surface on the backspan, the flat surface positioned to engage tissue after the tissue stapling.

16. The method of claim 10, further comprising:
providing a texturized surface on at least a portion of the surgical staple for promoting clotting of stapled tissue after the tissue stapling.

17. The method of claim 10, further comprising:
coating at least a portion of the surgical staple with a clotting agent.

18. A method, comprising:
providing a surgical stapler having an anvil opposing a cartridge, the cartridge housing at least one staple, the at least one staple having a pair of staple legs, a backspan positioned between the pair of staple legs, the backspan and the pair of staple legs formed by bending a wire, and at least one recess positioned in the wire, the at least one recess configured to influence a folding direction of at least one staple leg during tissue stapling, and wherein the surgical staple is configured to pierce tissue only once with each staple leg during the tissue stapling;
approximating the anvil and the cartridge with tissue positioned therebetween;
ejecting the at least one staple from the cartridge toward the anvil to cause the pair of staple legs pierce the tissue and to contact the anvil and bend the pair of staple legs inward to form the consistent tissue gap without piercing the tissue a second time.

19. The method of claim 18, wherein:
the backspan further comprises a flat surface positioned to engage tissue after the tissue stapling; and
the method further comprises positioning the flat surface against the tissue.

20. The method of claim 18, wherein:
the at least one staple has a texturized surface for promoting clotting of stapled tissue after the tissue stapling.

21. A surgical stapler, comprising:
an anvil opposing a cartridge, the cartridge housing at least one staple, the at least one staple having a pair of staple legs, a backspan positioned between the pair of staple legs, the backspan and the pair of staple legs formed by bending a wire, and at least one recess positioned in the wire, the at least one recess configured to influence a folding direction of at least one staple leg during tissue stapling, and wherein the surgical staple is configured to pierce tissue only once with each staple leg during the tissue stapling.

22. The surgical stapler of claim 21, further comprising:
a cutting mechanism having an elongated member having a length and a height, a proximal portion, a distal portion, and an elongated slot extending a portion of the length of the elongated member, wherein the distal portion comprises a cutting surface, and wherein the elongated slot comprises a distal slot portion, an intermediate slot portion, and a proximal slot portion, the distal slot portion positioned one of higher or lower than the intermediate slot portion, and the proximal slot portion positioned the other one of higher or lower than the intermediate slot portion; and
a first clamping member adjacent the distal portion of the elongated member.

23. The surgical stapler of claim 22, wherein
the distal portion of the elongated member is configured to detachably engage a second clamping member adjacent the distal portion of the elongated member.

24. The surgical stapler of claim 23, wherein
the second clamping member further comprises a sled configured to engage one or more staple pushers.
